(19) United States
(12) Patent Application Publication
(10) Pub. No.: US 2012/0316626 A1
(43) Pub. Date: Dec. 13, 2012

(54) THERAPEUTICE DEVICE

(76) Inventors: MaryCarol Dolivier, Chicago, IL (US); Arthur Blackwood, Derby (GB); Steve Pfister, Atlanta, GA (US)

(21) Appl. No.: 13/488,782

(22) Filed: Jun. 5, 2012

Related U.S. Application Data
(60) Provisional application No. 61/494,416, filed on Jun. 8, 2011.
(60) Provisional application No. 61/570,514, filed on Dec. 14, 2011.

(51) Int. Cl. A61F 7/08; A61F 7/10

(52) U.S. Cl. 607/108; 607/114; 607/112

(57) ABSTRACT

A therapeutic device for providing cold or heat therapy is provided. The device includes solid spherical materials and a medium that may include an alcohol mixture. An impermeable enclosure contains the spherical materials and the medium to form the therapeutic device. The therapeutic device applies cold or heat therapy and is pliable to conform to curved contours of the body. The therapeutic device also has a weight which provides pressure to, and desired contact with, the injured area. Certain embodiments further include an accessory for holding the device in contact or otherwise close proximity to a body region being treated.

### CROSS-REFERENCE TO RELATED APPLICATIONS

This application claims priority to U.S. Provisional Application Ser. Nos. 61/494,416, filed Jun. 8, 2011 and 61/570,514, filed Dec. 14, 2011, each of which is incorporated herein by reference in its entirety.

### TECHNICAL FIELD

Embodiments disclosed in the present application relate generally to thermal devices embodied as therapeutic hot/cold packs for treating physical discomfort.

### BACKGROUND

Everyday life provides a multitude of opportunities for injuries. Children may injure themselves during free play at school, in public parks, and at home. They may also bruise, sprain, or otherwise suffer injuries during recreational sports such as swimming, basketball, soccer, football, volleyball, and etc. Adults similarly suffer injuries from a whole range of activities, from routine house work (the stumped toe or bumped shin) to fitness workouts and recreational sporting.

Hot and/or cold therapy helps reduce or prevent the swelling and pain that accompanies tissue as well as musculoskeletal injury. Hot and/or cold therapy may also be used to reduce or prevent swelling and pain after surgical procedures (e.g., cosmetic surgery, broken bones, knee surgery, hip surgery, etc.).

Ice bags, gel packs, chemical cold packs and frozen vegetables are the most commonly used methods of delivering cold therapy. Ice bags are typically created by filling with refrigerator ice cubes a standard ZIPLOC, sandwich bag, grocery bag, or similar device. While the cooling effect of these bags last a long time, they tend to be difficult to acquire on the spot, for example, on a sporting field (where the cooler ice may already be melted or clumped into a mass), in a gym, or on a playground. Furthermore, ice bags often will not contour to the body part one desires to treat.

Cold gel packs become stiff when frozen—making them difficult or impossible to contour to the body—and may not provide a therapeutic amount of weight and/or contact to the region being treated. The manual manipulation required to operate and apply a cold gel pack may both aggravate the injured area, and may be impossible to perform by an injured person if that person is either alone or only has the use of one hand. Chemical cold bags may not produce the appropriate degree of cold and may contain materials that are toxic if ingested or applied to the skin after rupture.

Gel packs and commercial chemical ice packs may also contain toxic materials, such as acrylamide, which are skin and respiratory irritants. For example, acrylamide, a common ingredient in some commercially-available slush and gel packs, is an irritant and a carcinogen and a neurotoxin.

Heating pads and heated water bottles are the most common method of providing heat therapy. Also available are gel packs and chemical hot packs. Heated water bottles are typically used filling a rubber bladder with heated water. The heating effect of water bags and heating pads tend to be difficult to acquire on the spot, for example, on a sporting field or during travel (where the there is limited access to electricity to plug in a heating pad), in a gym, or on a playground.

Gel and chemical heat packs may not provide a therapeutic amount of weight and/or contact to the region being treated, and their heat may dissipate more quickly than desired such that the pack cools off more quickly than desired. Chemical hot packs may also contain materials that are toxic if ingested or applied to the skin after rupture. Gel packs may also contain toxic materials, such as acrylamide.

Additionally, there are very few devices that can be used to adequately and efficiently provide both hot and/or cold therapy. Most devices provide only one or the other. Those that do provide both are made of materials that degrade after only a few heatings and chillings/freezings and cannot withstand repeated use.

Many doctors recommend applying a bag of frozen vegetables to injured areas. Frozen vegetables are mobile and have a good malleability and weight, but are not reusable. Furthermore, frozen vegetables are not sanitary for carrying around in coolers at sports fields or in fitness centers, and the containers (e.g., bags) in which they come are not generally configured to be leak-proof during use as, for example, a cold-pack on an injured extremity.

### BRIEF SUMMARY

In one aspect, a therapeutic device for providing heat and/or cold therapy is provided. The device includes solid generally spherical materials suspended in an alcohol solution such as a polypropylene glycol solution. An impermeable enclosure contains the generally spherical materials and the alcohol to form the therapeutic device. The therapeutic device may be applied for heat and/or cold therapy and is pliable to conform to curved contours of the body. The device also has a weight which provides therapeutic pressure to the injured area.

In another aspect, an accessory for the therapeutic device is provided. The accessory includes the therapeutic device, a pouch for the therapeutic device, and a strap for attaching the accessory to the injured area. The accessory may be configured to be anatomically specific to fit the groin area of a human.

Other systems, methods, features, and advantages of the disclosure will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

FIG. 1 is a view of an exemplary therapeutic device;

FIG. 2A is a first view of an exemplary therapeutic device accessory;

FIG. 2B is a second view of an exemplary therapeutic device accessory;

FIG. 3 is a view of an exemplary therapeutic device assembled with an ankle;

FIG. 4 is a view of an exemplary therapeutic device assembled with a knee;

FIG. 5 is a view of an exemplary therapeutic device assembled with a wrist;

FIG. 6 is an exemplary therapeutic device with segments;

FIG. 7 is a view of an exemplary accessory with multiple pouches for use with a therapeutic device;

FIG. 8 is a view of an exemplary accessory with multiple pouches of varying sizes for use with a therapeutic device;

FIG. 9 is a view of an exemplary accessory for use with a therapeutic device;

FIG. 10 is a view of an exemplary accessory for use with a therapeutic device;

FIG. 11 is a view of one side of an exemplary accessory with multiple pouches for use with a therapeutic device;

FIG. 12 is a view of another side of an exemplary accessory for use with a therapeutic device;

FIG. 13 is a view of one side of an exemplary strap for use with a therapeutic device;

FIG. 14 is a view of another side of an exemplary strap for use with a therapeutic device;

FIG. 15 is a view of an exemplary anatomically specific accessory for use with a therapeutic device;

FIG. 16 is another view of the exemplary anatomically specific accessory for use with a therapeutic device;

FIG. 17 is an exemplary testing set up;

FIG. 18 is an exemplary temperature curve;

FIG. 19 is an exemplary temperature curve;

### DETAILED DESCRIPTION

A therapeutic device for providing heat or cold therapy is provided. The device includes solid generally spherical materials suspended in an alcohol solution. The generally spherical materials may be smooth, or may be textured, including—for example—that they may be regularly or irregularly faceted, and/or may include ridged or otherwise convoluted surfaces. Heat and cold therapy may reduce or prevent the swelling and pain that may accompany injuries, for example, injuries caused by sporting accidents, fitness routines, accidents that occur during play, and otherwise. Heat and cold therapy may also reduce or prevent swelling and pain that accompanies the post surgical healing processes.

Heat and/or cold therapy may be applied after injury to help relieve the pain and keep the area from swelling. When there is an injury, fluid may collect in the injured area and cause swelling and pain. Heat and/or cold therapy may be used for injuries, for example but not limited to bruises, sprains, strains, and pulled muscles. Heat and/or cold therapy may also be used for pain related to menstruation, arthritis, migraines, and otherwise.

Health and first aid professionals may suggest the use of a one-pound package of frozen corn or peas (or other similarly dimensioned frozen fruit, grain, or vegetable) to make an ice pack. These professionals cite characteristics such as being lightweight, and the ability of the package of frozen corn or peas to conform to the injured area. The cold therapy may be optimal when used, for example, for about 10—about 20 minutes. However, as those of skill in the art (and any prior user) will appreciate, the sensory therapeutic value of frozen food items tends to degrade over time as the materials/objects in the bag tend to become mushy as the cold dissipates. This diminishes the multi-point/multi-surface contact and the “flowability” associated with tactile sensation of the solidly-frozen items in the bag moving against each other and the user's body. Even devices that have attempted to mimic the different appealing properties of a bag of frozen peas may fall short by being stiffer, less compliant, less conforming, and/or having less desirable tactile flowability as compared to the traditional “bag of peas.” Some products may include soft, compliant, or otherwise non-rigid/non-solid items within a container that do not provide the tactile sensation desired and provided by the present embodiments. Some of those other products also are configured only to be heated or only cooled, but not configured for a single product to be heated and/or cooled. For example, some products containing free-flowing or encapsulated gel material may be useful only for cold application, without providing the composition and function of the presently-disclosed embodiments that allow heating or cooling in turn, as desired by a user (e.g., a device of the present disclosure may be used out of the freezer several times, heated up for use several times, etc. in whatever order needed/desired by a user). Embodiments disclosed herein retain the advantages and tactile/sensory desirability associated with one's first contact with a traditional “bag of peas,” while providing superior performance with regard to time remaining cold without degradation of desirable tactile sensation, conformance to body surface, and the ability to provide heat with the same desirable tactile sensation and body-surface conformity associated therewith.

Use of some pressure or compression may also help prevent or reduce swelling. It may be important that pressure is applied to the area without cutting off circulation to the area. Some studies have shown that cold therapy with some compression increases the rate of heat transfer from the injured area into the cold pack—which may reduce swelling and thus pain at an increased rate. Many users may also find desirable the many small rounded points of contact with the generally spherical solids, which may provide a massage-like effect and/or other desirable tactile stimulation/sensation.

The disclosed therapeutic device may have features of producing effective but safe degree of (alternatively) heat and/or cold over an extended period of time while also having the added features of being more flexible and contoured than alternatives, reusable, less toxic than alternatives, and also more cost-effective and efficient to manufacture than alternatives. The disclosed therapeutic device may comprise at least about 3 simple elements. It may be uncomplicated to assemble and manufacture. The therapeutic device may also provide some weight to the injury so that additional compression devices are not required. The disclosed therapeutic device may be used more than once per freezing and/or heating and it may provide at least an even, smooth, cold therapeutic effect for at least about 10-20 minutes, without the chilled solid objects that provide desirable tactile sensation becoming softened or otherwise degraded in a manner that could negatively impact therapeutic value and user-desired sensation.

The disclosed therapeutic device may be applied to animals as well as humans to treat both injuries caused by external factors, (e.g., sprains, bruises, breaks, etc.) and by internal factors (e.g., arthritis). For example, the device may be used to treat pets, recreational animals, and work animals for injury, exertion, and/or other conditions that may be alleviated by application of heat and/or cold in a manner conforming to a treatable body surface.

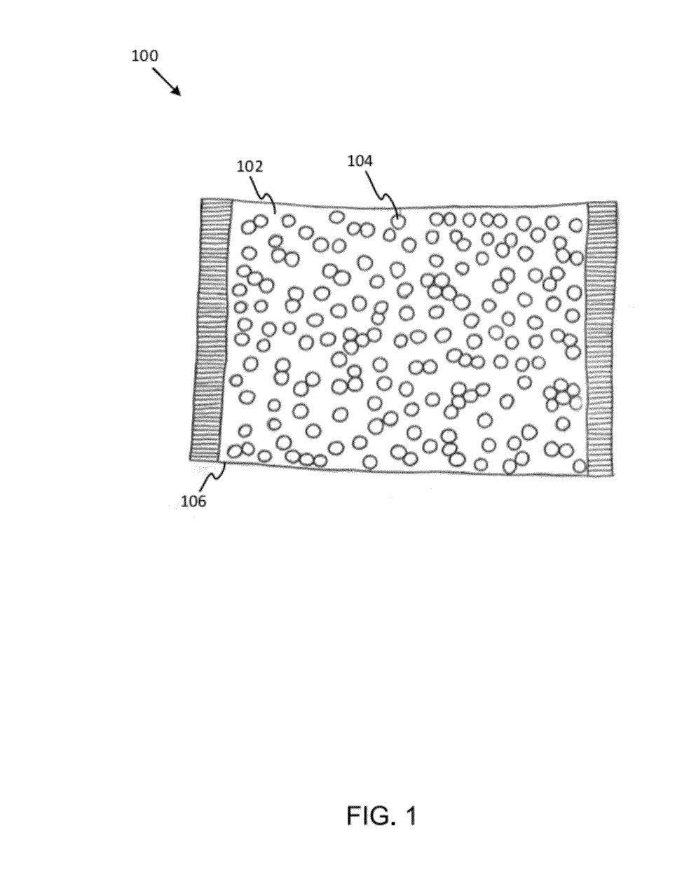

One embodiment of a therapeutic device 100, which may be a therapeutic device for providing heat and/or cold therapy, is illustrated with reference to FIG. 1. A therapeutic device 100 may include at least a packaging 106. The packaging 106 may be made of one or more layers of material. For example, the packaging 106 may be made of a flexible, insulating material. The packaging 106 may help extend effective heat and/or cold therapy time by minimizing heat gain from the environment. Alternatively or additionally, the packaging 106 may be made of a flexible material that provides little or no insulating properties (i.e., its construction does not significantly affect thermal transfer in a manner preserving or accruing heat to the materials within the packaging).

The packaging 106 may include a medium 102 and discrete solid materials 104. The discrete solid materials 104 may be spherical, ellipsoidal, or any other shape. For example, the discrete solid material 104 may be a faceted sphere or ellipsoid, a generally spherical solid with indentations or holes, or any other shape with faceted faces, faces with indentations or holes, including any variant or combination thereof. The discrete solid materials 104 may be solid throughout, containing no inner compartments, fluids, or otherwise. For example, the discrete solid materials 104 may be at least one of glass (e.g., borosilicate glass, Grade A, B, or C soda-lime glass, or other), granite, soap stone, steel, clay, ceramic (glazed or unglazed), high-density plastic or other injection molded material, any other thermal retaining material, or any combination thereof. The discrete solid materials 104 may contain air pockets and/or natural impurities or those impurities inherent, accidentally, or intentionally introduced in manufacturing processing, mining, milling, or otherwise and may still be considered solid, by which is meant generally or substantially rigid and non-compliant.

The discrete solid materials 104 may be spherical balls. For example, the discrete solid materials 104 may be glass balls, granite balls, soapstone balls, high-density plastic balls, hard (substantially non-deformable) rubber balls, or otherwise. In certain embodiments of the therapeutic device, the discrete solid material 104 may be spheres (i.e., at least generally spherical bodies) with diameters in the range of about 4 mm to about 14 mm, with certain preferred embodiments having diameters in the range of about 6 mm to about 12 mm. In certain devices, the spheres may have a substantially uniform diameter of about 6 mm; in other devices, they may have a substantially uniform diameter of about 9 mm; and, in certain embodiments, spheres of different diameters may be included. Certain preferred embodiments may have spheres with a range of diameters in a single device. Other preferred embodiments may have spheres with a substantially uniform diameter that may vary from a mean diameter by 2 mm or less.

The medium 102 may be an alcohol solution. The medium 102 may include, for example, at least one of glycerin, water, alcohol, propylene glycol, and/or propane-1,3-diol (the latter two of which, as a “double alcohols,” are included in the definition of the term “alcohol” for purposes of this disclosure). The medium may be substantially in a fluid phase. A fluid phase may refer to a phase, such as a gas or a liquid, which has no fixed shape and yields easily to external pressure. Furthermore a fluid phase may indicate the property of the medium to flow and change its shape at a steady rate when acted on by a force tending to change its shape and also the property of taking the form of the container that it is in. A fluid phase may contain some solids, yet be at least about 60% fluid and/or liquid. In other words, the medium 102 may contain some ice crystals and still be considered fluid phase. The medium 102 may include, for example, a combination of an alcohol (including, as defined above a double alcohol such as—for example—propane-1,3-diol or propylene glycol) and water which may be at a ratio of 5:95, 10:90, 20:80, 25:75, 50:50, 65:35, 70:30, 75:25, 80:20, 90:10, 95:5 or other ratios or other intervening ratios, for example but not limited to about 27.5:72.5. In another embodiment, the medium 102 may include, for example, a combination of glycerin and alcohol, which may be at a ratio of 5:95, 10:90, 20:80, 25:75, 50:50, 65:35, 70:30, 75:25, 80:20, 90:10, 95:5, or other ratios or other intervening ratios, for example but not limited to about 27.5:72.5.

In another embodiment, the medium 102 may include, for example, a combination of propylene glycol and another alcohol, which may be at a ratio of 5:95, 10:90, 20:80, 25:75, 50:50, 65:35, 70:30, 75:25, 80:20, 90:10, 95:5, or other intervening ratios, for example but not limited to about 27.5:72.5. In another embodiment, the liquid may include, for example, a combination of propylene glycol and water which may be at a ratio of 5:95, 10:90, 20:80, 25:75, 50:50, 65:35, 70:30, 75:25, 80:20, 90:10, 95:5, or other ratios and/or other intervening rations for example but not limited to about 27.5:72.5. In another example, if the alcohol is glycerine or polyglycerol the concentration of glycerin or propylene glycol may be between approximately 1%-approximately 50%, approximately 5%-approximately 40%, and/or approximately 15%-approximately 40%. In one example, the medium may include a solution that is about 30% propane-1,3-diol (available under the trade name Zemea™) and 70% water occupying about 85% of a device volume, with the remaining about 15% occupied by soda-lime glass beads.

The medium 102 may maintain viscosity at low temperature such that the therapeutic device 100 remains malleable. For example, the therapeutic device 100 may have the property of being formable to contoured surfaces by virtue of, for example but not limited to, the weight of the discrete solid materials 104 relatively unencumbered by the viscosity or solidity of the medium 102. In another embodiment, the therapeutic device 100 may have the property of being formable to contoured surfaces by virtue of, for example but not limited to, the weight of the discrete solid materials 104 relatively unencumbered by the viscosity or solidity of the medium 102 such that little or no manual manipulation is required to contour the therapeutic device 100 to a contoured surface. In another embodiment, the therapeutic device 100 may closely approximate, for example but not limited to, a bag of frozen peas.

The medium 102 may be, for example, but not limited to, a composition of water and glycerin, a composition of water and alcohol, a composition of glycerin and alcohol, a composition of propylene glycol and alcohol, a composition of propylene glycol and water. The liquid may alternatively or additionally contain only water, only alcohol, only propylene glycol, or only glycerin. The therapeutic device 100 may contain other materials such as cellulose or sugars, colloids, or otherwise and also may contain antibiotic or other anti-spoil agents. Alternatively, in some embodiments, the therapeutic device 100 may contain only a medium 102 made of three or fewer components, a package 106, and solid discrete materials 104.

### Example 1

### PRODUCT TYPE: PE/PA/PE and PE/PA

### WEIGHT: Approx. 860 Grams

### DIMENSIONS: 7″ by 6″

### No. of Balls: Approx. 550

### Volume of Liquid: 300 ml

### Dia. of Balls: 9 mm

Film Thickness: 90 and 100 micron
Liquid Mixture Ratio: 72.5 (water):27.5 (alcohol, such as, but not limited to, glycol, glycerine, propylene glycol, propane-1,3-diol, or a petroleum free glycol alternative)

### Example 2

### Weight: 850 g

### Dimension: 7.5×6

No. of Balls: approx. 646

### Volume Liquid: 250 ml

### Example 3

### THREE POCKET BAG:

### PRODUCT TYPE: PE/PA/PE and PE/PA

### WEIGHT: Approx. 1450 Grams

### DIMENSIONS: 15″ by 6″

### No. of Balls: Approx. 1000

### Volume of Liquid: 450 ml

### Dia. of Balls: 9 mm

Film Thickness: 90 and 100 micron
Liquid Mixture Ratio: 72.5 (water):27.5 (alcohol, such as, but not limited to, glycol, glycerine, propylene glycol, propane-1,3-diol, or a petroleum free glycol alternative)

### Example 4

### EYE PACK—SINGLE POCKET

### PRODUCT TYPE: PE/PA/PE and PE/PA

### WEIGHT: Approx. 270 Grams

### DIMENSIONS: 7″ by 2.75″

### No. of Balls: Approx. 180

### Volume of Liquid: Approx.100 ml

### Dia. of Balls: 6 mm

Film Thickness: 90 micron
Liquid Mixture Ratio: 72.5 (water):27.5 (alcohol, such as, but not limited to, glycol, glycerine, propylene glycol, propane-1,3-diol, or a petroleum free glycol alternative)

The therapeutic device 100 may substantially resist accumulation of the internal materials (e.g., the medium 102 and the solid discrete materials 104) without the need for internal compartments. The therapeutic device 100 may substantially resist accumulation of the internal materials (e.g., the medium 102 and the solid discrete materials 104) without the need for management of air. For example, the therapeutic device 100 may include no internal air. Alternatively or additionally, the therapeutic device 100 may be configured to include internal seams such that the device is more easily wrapped around an object, such as an animal or human limb.

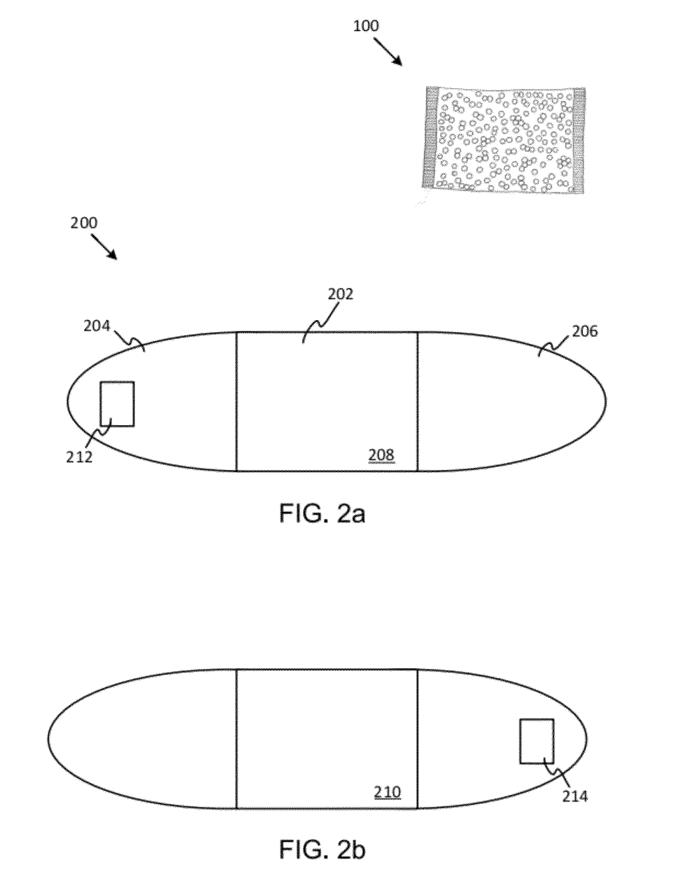

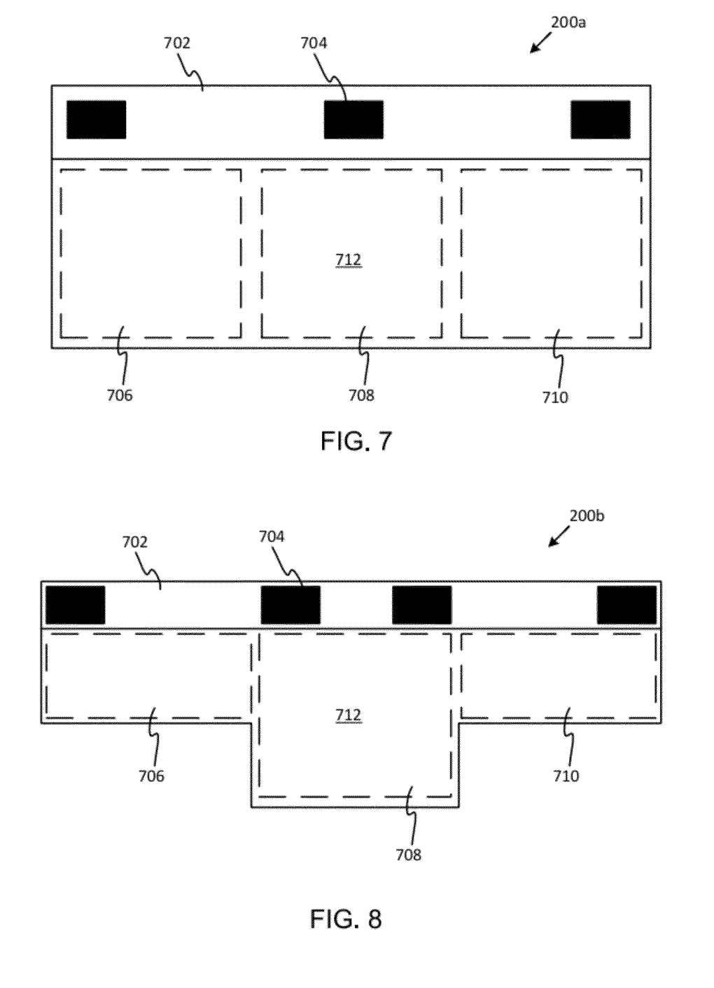

Another embodiment of a therapeutic device 100 which may be used with an accessory 200, is illustrated with reference to FIG. 2 (with variant embodiments described below, referring to FIGS. 7-16). An accessory 200 may include an elongate material portion that may have at least three sections 202, 204, 206. In some embodiments, a pouch section 202 may include a pocket or pouch that may be dimensioned to hold a therapeutic device 100. In FIG. 2, the section 202 is shown as a middle section; however, the section 202 may be located anywhere on the accessory 200. Additionally or alternatively, the accessory 200 may have multiple pockets or pouches.

The accessory 200 may also include a first wrap section 204 and a second wrap section 206. The first wrap section 204 and the second wrap section 206 may (each and/or both) include attachment means, 212, 214. In this example, the attachment means 212, 214 are located such that attachment may occur/be engaged when the accessory 200 is wrapped around a contoured surface. Attachment means 212, 214 may include a hook and loop fastener, such as Velcro™ (including, for example, Velcro™-type hook surface(s) and a fabric such as flannel or a Velcro™-compatible loop backed fabric like neoprene that will engage with such), magnet(s), button(s), and/or snap(s), or any other means of attachment now known or yet to be developed, each without exceeding the scope of this disclosure. Further, attachment means 212, 214 may be otherwise located.

The accessory pouch section 202 may have a first surface 208 and a second surface 210. The first surface 208 may be a surface that faces the skin. The first surface 208 may be made of mesh, Neoprene, cotton, rubber, flannel, or a similar material that will allow thermal transfer between the contoured surface (e.g., a leg, arm, wrist, or otherwise) and the therapeutic device 100. Preferred materials on surfaces intended to contact a user will include user-desired skin-feel (e.g., soft flannel, brushed cotton, silk, etc.). Preferred surface materials in certain embodiments may include or even consist of hypoallergenic materials to minimize likelihood of skin irritation. The material preferably will also help to provide comfort by assisting in regulation of thermal transfer. By this, it is meant that—when the device is cold—a preferred material facing the user may limit to sensation of coldness due to thermal transfer out of the user to a range that is comfortable, thereby minimizing or avoiding a “too cold on my skin” sensation; conversely,—when the device is heated—a preferred material facing the user may limit to sensation of heat due to thermal transfer to the user to a range that is comfortable, thereby minimizing or avoiding a “too hot on my skin” sensation. The second surface 210 may face away from the skin and may be made of a material that has insulating properties. Additionally or alternatively, the second surface 210 may be made of materials such as Neoprene, cotton, rubber, flannel, or otherwise.

The accessory 200 may assist in holding the therapeutic device 100 in a location. Alternatively or additionally, the accessory 200 may provide added compression. The accessory 200 may include extension devices that allow the accessory 200 to fit various dimensioned surfaces.

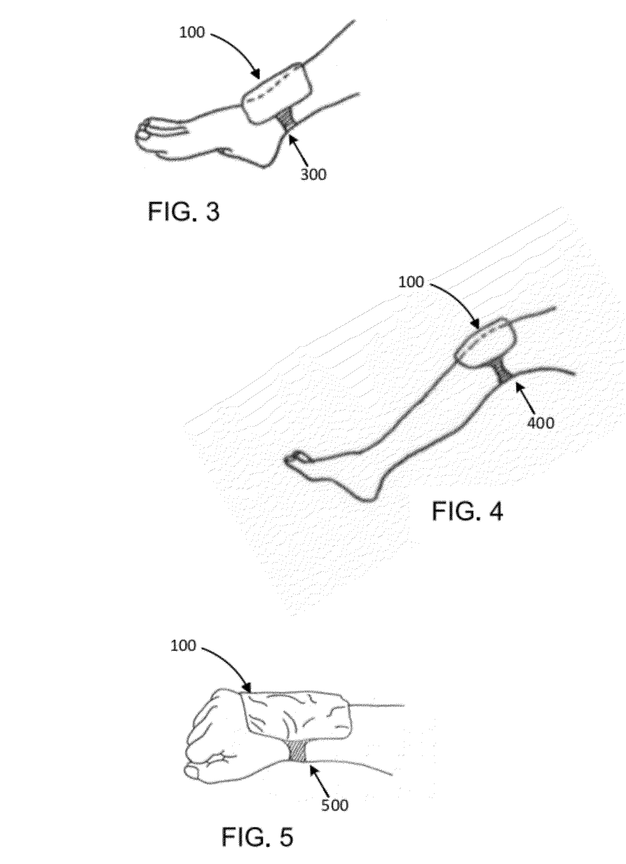

In yet another embodiment, the therapeutic device 100 may be used on a contoured surface such as an ankle 300, knee 400, or wrist 500, as illustrated with reference to FIGS. 3, 4, and 5, respectively. Although we only illustrate some exemplary surfaces, those of skill in the art will appreciate that other body regions and/or surfaces may be treated with various embodiments, including as described below.

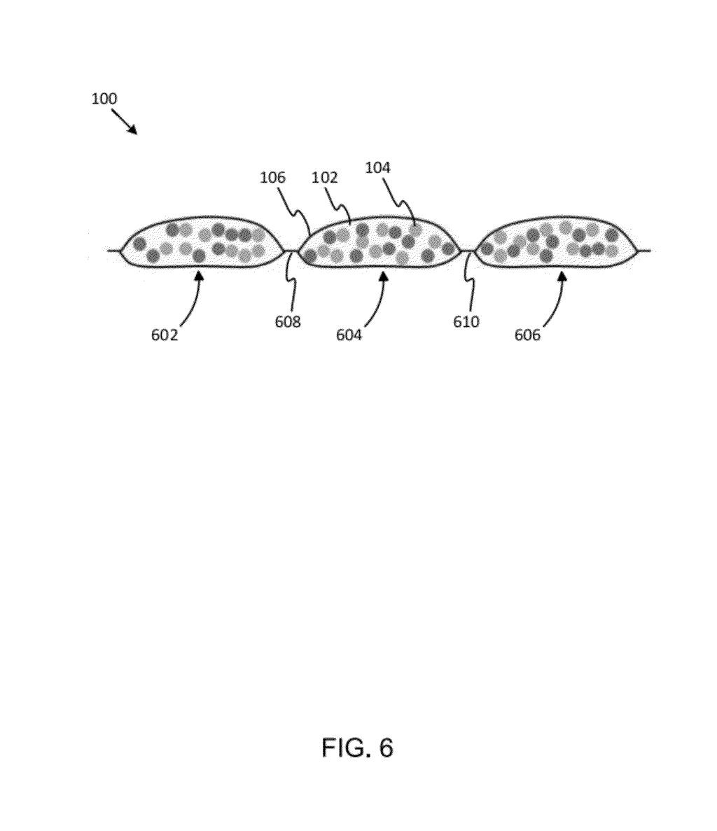

An embodiment of the therapeutic device 100 may include seam-separated or otherwise distinct segments 602, 604, 606 such that the therapeutic device may be more easily wrapped around an injured area, as illustrated with reference to FIG. 6. The therapeutic device 100 may include any number of segments, and the segments may be square, rectangular, round, or any other shape. The segments may have the same dimensions or varying dimensions. They may be separated from each other by dividing portions 608, 610, which may be embodied as impermeable seams (e.g., of the container material) or by seams configured to allow fluid communication of the medium therebetween. In various embodiments, communication between segments of medium and/or solids may be controlled by dimensioning the material between the segments.

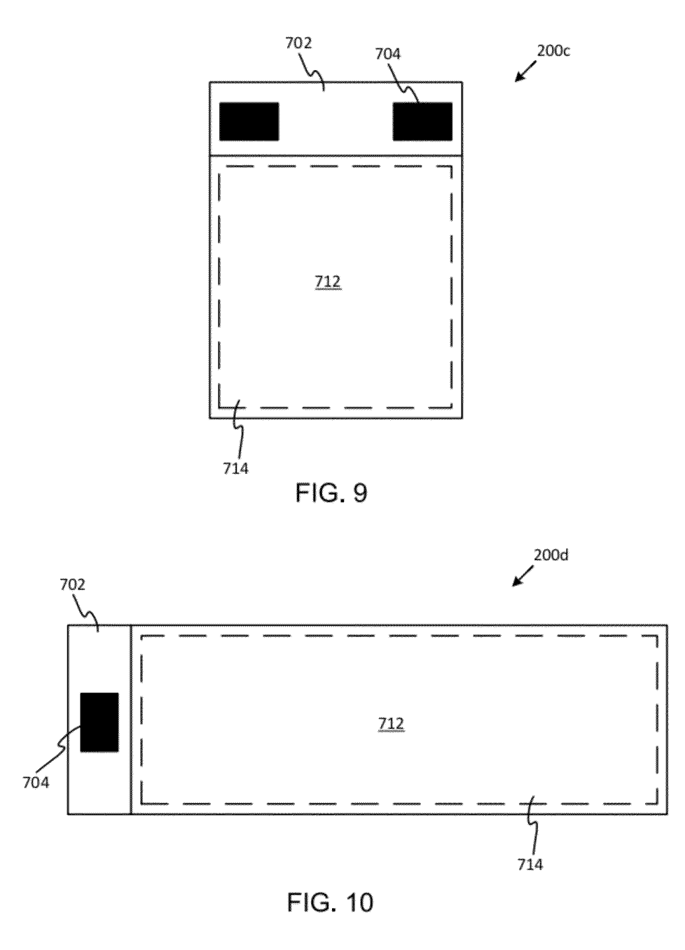

Additional embodiments of the therapeutic device 100, which may be used with varied constructions of an accessory 200 (shown as 200a, 200b, 200c, 200d, 200e and 200f by way of non-limiting example), are illustrated with respect to FIGS. 7-10. An accessory 200a may have a flap 702 that folds over the pouch section to hold a therapeutic device 100 in the pouch section. The flap 702 may include any flexible material, or combination of materials, including but not limited to Neoprene, cotton, rubber, or flannel, or any flexible organic or synthetic material, as described herein and/or otherwise known in the art. Attachment means 704 may be provided on the flap 702 to allow removal and replacement of the therapeutic device 100. Attachment means 704 may be constructed and implemented in a variety of manners, including those referenced elsewhere herein. The number and placement of the attachment means 704 may vary depending on the size and shape of the accessory 200 and the therapeutic device 100. An accessory 200 may have any number of pouch sections. For example, an accessory 200 may have multiple accessory pouch sections 706, 708, 710 as shown in FIG. 7 (200a) and FIG. 8 (200b). The first surface 712 of the pouch section may be any material, or combination of materials, that allows thermal transfer between the surface of an injured area and the therapeutic device 100. For example, the first surface 712 may be made of mesh, Neoprene, cotton, rubber, flannel, or otherwise. As shown in FIG. 7, the multiple pouch sections 706, 708, 710 may be the same size. Alternatively, or additionally, as shown in FIG. 8, the multiple pouch sections 706, 708, 710 may be different sizes. Alternatively, an accessory 200 may have a single pouch section 714 as shown in FIG. 9 (200c) and FIG. 10 (200d). Each pouch section 706, 708, 710, 714 may be sized and/or shaped to hold one or more therapeutic devices 100.

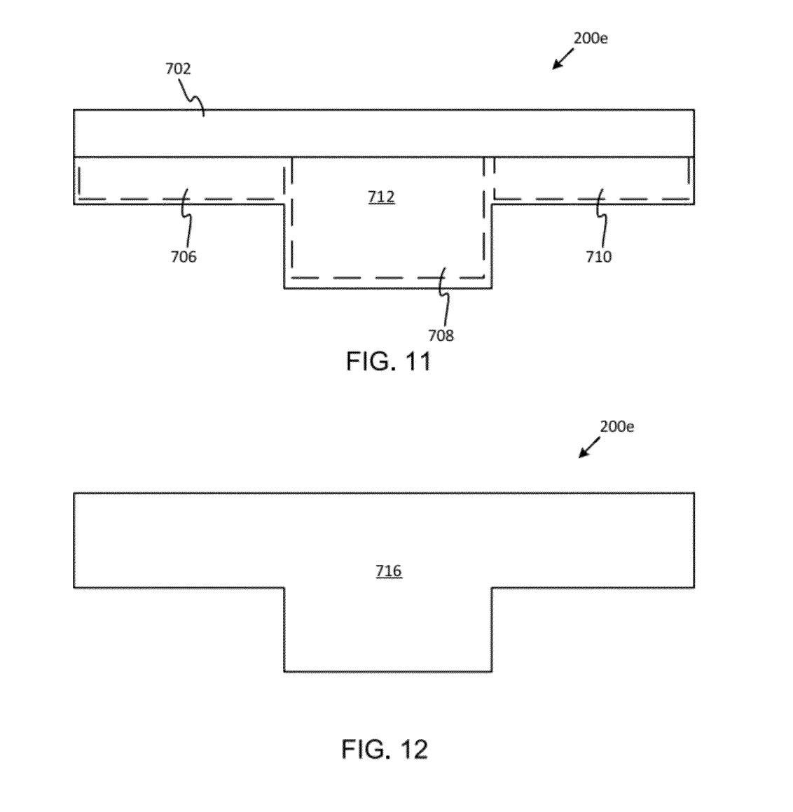

As shown in FIGS. 11 and 12 (showing opposite sides of a pouched structure), an embodiment of the therapeutic device 100 may be used with an accessory 200e including a flap 702 that folds over the pouch section. The first surface 712 of the accessory 200e is shown in FIG. 11 and the second surface 716 of the accessory 200e is shown in FIG. 12. The first surface 712 may be a surface that faces the skin of the injured area, and the second surface 716 may face away from the skin. The second surface 716 may be made of the same or different material as the first surface 712. For example, the second surface 716 may be made of mesh, Neoprene, cotton, rubber, flannel, or otherwise. The second surface 716 may be customized with any variety of colors, patterns, prints, pictures, emblems, logos, buttons, pins, and/or patches. For example, an injured sports player may choose an accessory 200 that has the team colors or team emblem of the team that the player represents. As another example, children may prefer an accessory 200 with bright colors or cartoon characters printed on the second surface 716. It should be appreciated that such color(s), decoration, logo-placement, and other visual, embossed, or other adornment or indicia may be used with and/or incorporated into other embodiments of accessories. Additionally, or in the alternative, such adornment or indicia may be included in embodiments of the device 100. For example, the packaging 106 (and/or other suitable outer container) may include colors, pictures, logos or the like; the generally spherical solids and/or the medium may include colors, designs, or other decorative or functional indicia.

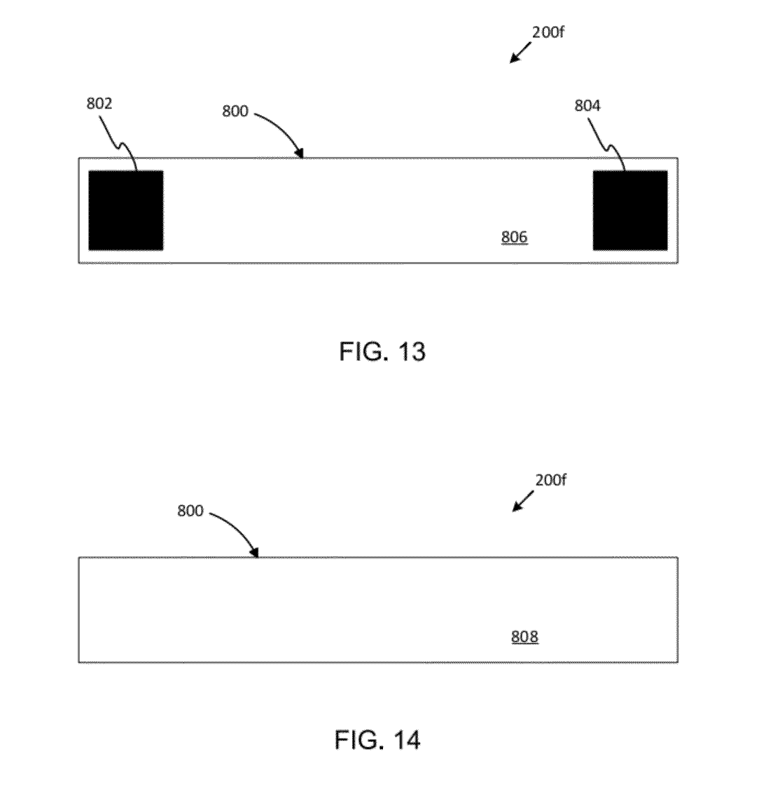

In another embodiment, the therapeutic device 100 may be used with an accessory 200f that includes a strap 800 which may be used to hold the accessory 200f in place over the injured area, as illustrated with reference to FIGS. 13 and 14, which show opposite sides of the accessory. The strap 800 may have attachment means 802, 804 on each end. Alternatively, or additionally, the attachment means 802, 804 maybe positioned at any other location(s) along the strap 800. Other embodiments of the strap 800 may have fewer or additional attachment means positioned at various locations along the strap. The strap 800 may be made entirely of elastic material, or may have sections of elastic material and sections of fabric, such as, but not limited to, nylon, Neoprene, cotton, or otherwise. Attachment means 802, 804 may be constructed and implemented in a variety of manners, including those referenced elsewhere herein. The strap 800 may be attached or sewn onto the accessory 200, or may be completely detachable from the accessory 200. One or more straps 800 may be positioned in any orientation or location that will hold the accessory securely to the injured area. The first surface 806 of the strap, as shown in FIG. 13, may be a surface that faces the skin of the injured area. The second surface 808 of the strap, as shown in FIG. 14, may face away from the skin. The second surface 808 may be customized with any variety of colors, patterns, prints, pictures, emblems, logos, buttons, pins, and/or patches in this and/or other embodiments.

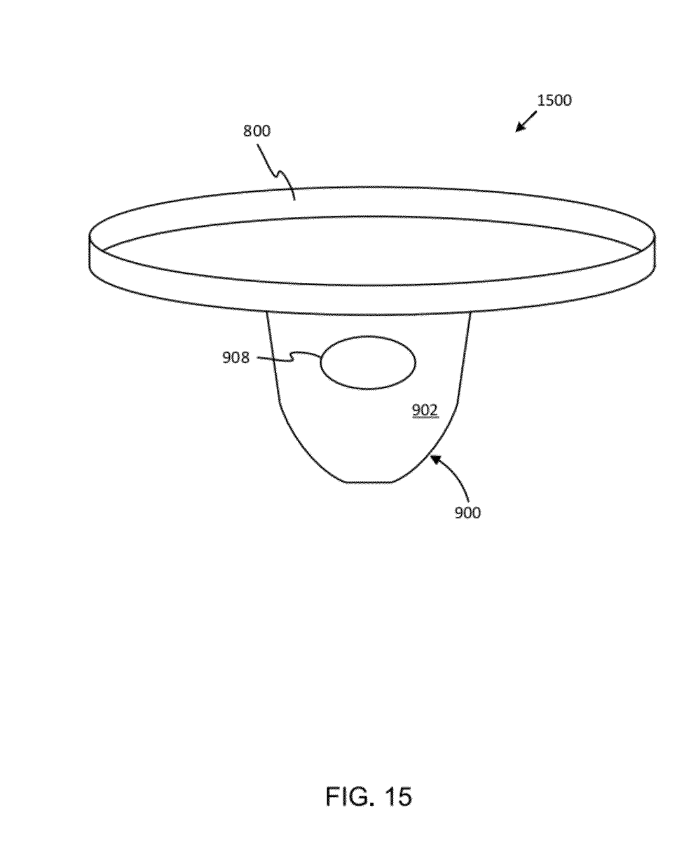

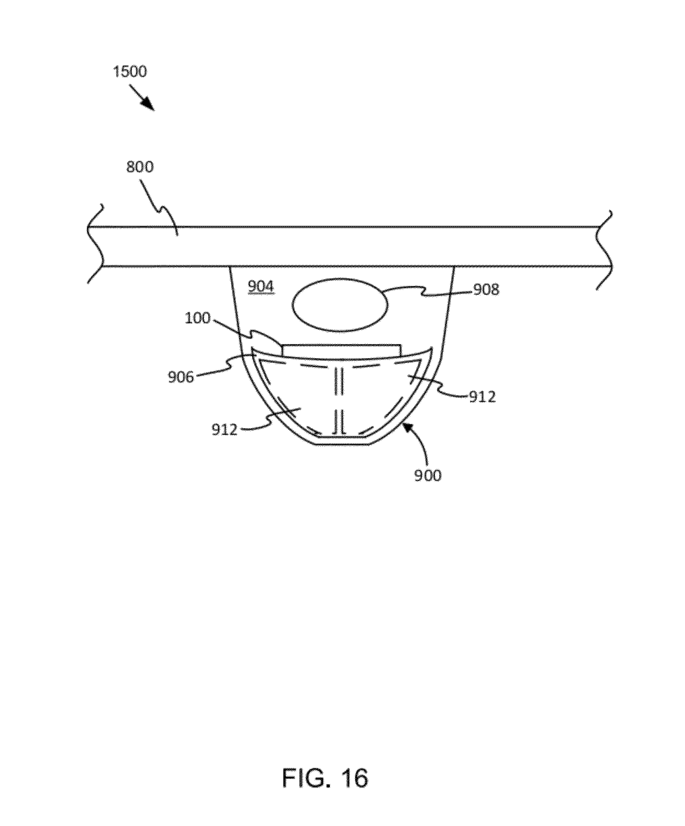

In yet another embodiment, the therapeutic device 100 may be used with an anatomically specific accessory 1500, as illustrated with reference to FIGS. 15 and 16. In this embodiment, the accessory 1500 may include a strap 800 that is sized to fit around the waist or other body portion of a person so as to hold a therapeutic device 100 over an injured area. The accessory 1500 may be configured to be anatomically specific for treatment of an injury to the groin or scrotum/testes of a wearer. For example, the accessory 1500, as shown in FIGS. 15 and 16, may be used for post-vasectomy or other post-surgical treatment of the groin or scrotum area of the wearer, as is commonly medically recommended.

The accessory 1500 of FIGS. 15 and 16 may be used to treat an injury to the groin area or scrotum/testes, such as, but not limited to, a pulled muscle, a contusion or any injury resulting, for example, from sports or other activities. The pouch section 900 may be removably attached to the strap 800 by loops, a hook and loop fastener, or other attachment means. Attachment means may be constructed and implemented in a variety of manners, including those referenced elsewhere herein. The pouch section 900 may also include one or more other attachments to the side and/or rear of the strap 800 (e.g., in a manner constructed similar to an athletic supporter or an undergarment generally constructed as briefs). Alternatively or in addition, the pouch section 900 may be sewn to the strap, or otherwise attached to the strap.

The pouch section 900 may be sized and shaped to fit the groin area of a person. The embodiment shown in FIGS. 15-16 is configured for treating a male user in need of cold or heat applied to the groin, scrotum/testes, and/or penis, but may also be configured to a similar design for a female user (e.g., to apply a device 100 as a cold-pack post-episiotomy or to otherwise treat the perivaginal region for post-natal discomfort or other discomfort). A first surface 902 of the pouch section 900 may be a surface that faces away from the skin of the area to be treated. A second surface 904 of the pouch section 900 may face the skin of the area to be treated. The second surface 904 (or the opposite surface) may have a holding area/pocket 906 for inserting the therapeutic device 100. An optional window 908 may be provided to allow the user access to his penis (e.g., for urination) without needing to move the rest of the pouch in a manner dislodging the device 100 from its desired location.

In one preferred embodiment, the pocket 906 is dimensioned and located on the pouch for placement of a device 100 that will keep it in contact with a region of the scrotum/testes subject to inflammation post-vasectomy. The pocket 906 and/or the pouch section 900 may be constructed to allow a user/wearer to position the device 100 therein at least partially dorsal of the scrotum (e.g., contacting its underside and/or between the scrotum and perineum). In another embodiment, the pocket 906 may be located to contact an incision site and/or region impacted by hernia-repair surgery. Velcro, stitching, or other means may be used to create two or more compartments within the pouch 906, where the compartments allow a user to orient the device 100 to be kept in close contact with a specific region (e.g., of the scrotum/testes over an incision, puncture, or impact site). Two such compartments are delineated with dashed lines 912, but those of skill in the art should appreciate that such compartments may be delineated in a variety of shapes, sizes, and orientations/locations within the scope of the present embodiments. Whether used with or without a strap 800, the pouch may be worn with a user's regular undergarments. In some embodiments, the pouch may be incorporated into an undergarment such as briefs, boxers, bikini-style underpants, an athletic supporter (a/k/a “jock strap”) or other garment configured to hold the pouch in close proximity and/or contact with a body region being treated with the device. Any such incorporation may be by stitched assembly, or by user application (e.g., via manual positioning, which may include securing with adhesive, Velcro, or other attachment means).

Although several accessory embodiments 200, 1500 are disclosed, such an accessory is not necessary for the proper use or application of the therapeutic device 100. The therapeutic device 100 may be used with or without straps, fasteners, elastics, and other similar means.

### Experimental Example

The following example illustrates some of the properties of certain embodiments of the disclosed invention and do not limit the scope of the invention described in the claims.

Test Setup

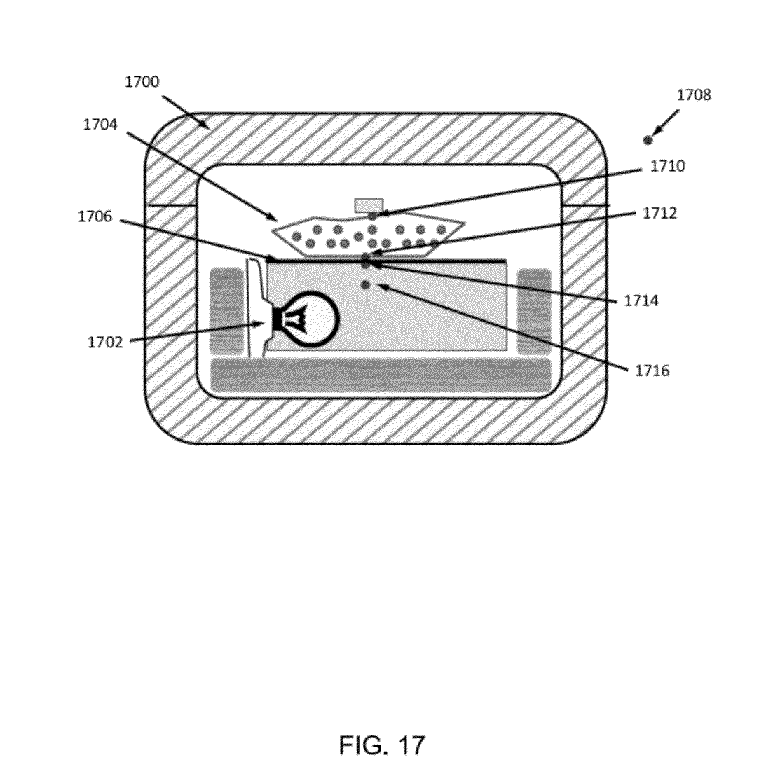

A set up was prepared as shown in FIG. 17. The test rig may comprise 1700 an insulated testing vault such as a Styrofoam cooler, a heat source 1702, such as a 100W light bulb, a sample 1704 for example, a commercial cold pack or the therapeutic pack as disclosed herein, and a conductive plate 1706. Measurements may be taken at thermocouples, for example, a thermocouple 1708 may measure room temperature, thermocouple 1710 may measure temperature of the sample 1704, thermocouple 1712 may measure temperature at the top of the conductive plate 1706, thermocouple 1714 may measure temperature at the bottom of the conductive plate 1706, and thermocouple 1716 may measure the temperature in the air space below the conductive plate 1706.

Test setup may include the following steps: 1) Soak ice pack test samples in freezer at −8.5+/−1° F. (−22.5+/−0.5° C.) for 6 hours, 2) Soak test rig at room temperature 75° F.+/−2° F. (24+/−1° C.) for one hour with plate removed and fan blowing air around interior to ensure that the entire rig is saturated at room temperature, 3) Setup data acquisition for recording data every 10 seconds (0.10 Hz), 4) Check that all thermocouples are calibrated and operating properly, and 5) Attach Thermocouples where shown.

Test Procedure

The test procedure may include the following steps: 6) Start Data Acquisition recording thermocouple temperatures, 7) Record 10 minutes of stabilization of test rig temperatures within 3° F. (+/−1.5° F.) range at room temperature (Test rig & ambient temperatures within range for 10 minutes.),8) Remove the test cold pack from the freezer and attach thermocouple 1710 with foam insulating tape near the center top side of the package, 9) Place the cold pack on the top center of the plate as shown and turn on power to heater bulb, 10) Close the insulated box with the insulated cover, 11) Steps 8 through 10 should be completed within 15 seconds, 12) Turn off bulb after 30 minutes or until any temperature exceeds safe limits, and 13) Collect data for another few minutes and save data file.

The following Table demonstrates exemplary known products:

| | | | | | | Heat |
|---|---|---|---|---|---|---|
| | | | | | | Capacity |
| Sample | Description | Dimensions | Weight | Solid | Medium | mCp |
| 1 | frozen peas | 5.5 × 7″ | 0.35 kg | peas | N/A | 568 J/K |
| | (food stuff) | 38.5 sq.in. | (.75 lbs) | | | (1153 J/K |
| | | | | | | melted) |
| 2 | ALLEGIANCE | 4 × 9.5″ | 0.32 kg | N/A | Thermal | — |
| | Gel Pack | 38 sq.in. | (.70 lbs) | | gel | |
| 3 | 3M Gel Pack | 4 × 9.5″ | 0.204 kg | N/A | Thermal | — |
| | | 38 sq.in. | (0.45 lbs) | | gel | |
| 4 | Glass balls | 6 × 6.5″ | .43 kg | Glass | N/A | 363 J/K |
| | only | 39 sq in. | (0.95 lbs) | balls | | |
| 5 | Glass & | 6 × 6.5″ | .58 kg | Glass | Glycerin | 722 J/K |
| | Glycerin | 39 sq in. | (1.28 lbs) | balls | .148 kg | |
| | | | | 8 mm | (.326 lbs) | |
| | | | | .43 kg | | |
| | | | | (0.95 lbs) | | |
| 6 | Glass & | 6 × 6.5″ | .57 kg | Glass | Glycerin | 830 J/K |
| | Glycerin | 39 sq in. | (1.26 lbs) | balls | .22 kg | |
| | | | | 8 mm | (0.49 lbs) | |
| | | | | .35 kg | | |
| | | | | (0.76 lb) | | |
| 7 | Glass & | 6 × 6.5″ | 0.58 kg | Glass | Alcohol | 692 J/K |
| | Alcohol | 39 sq in. | (1.28 lbs) | balls | 0.137 kg | |
| | | | | 8 mm | (0.30 lbs) | |
| | | | | .43 kg | | |
| | | | | (0.95 lbs) | | |
| 8 | Steel & | 6 × 6.5″ | 0.53 kg | Steel | Glycerin | 975 J/K |
| | Glycerin | 39 sq in. | (1.17 lbs) | balls | 0.182 kg | |
| | | | | D9.5 mm | (0.401 lbs) | |
| | | | | .89 kg | | |
| | | | | (1.96 lbs) | | |
| 9 | Glass & | 6 × 6.5″ | 0.53 kg | Glass | Prop Gl. | 777 J/K |
| | Propylene | 39 sq in. | (1.17 lbs) | balls | 0.182 kg | |
| | Glycol | | | 8 mm | (0.401 lbs) | |
| | | | | .346 kg | | |
| | | | | (0.76 lbs) | | |
| 10 | Glass & | 6 × 6.5″ | 0.735 kg | Glass | Prop Gl. | 1225 J/K |
| | Propylene | 39 sq in. | (1.62 lbs) | balls | 0.303 kg | |
| | Glycol | | | 8 mm | (0.669 lbs) | |
| | | | | .432 kg | | |
| | | | | (0.95 lbs) | | |

In this non-limiting example the liquids for samples 5-11 were the following:

| Liquid | Composition |
|---|---|
| Glycerin | 99.5% glycerin |
| Alcohol | 70% isopropyl alcohol |
| | 30% water |
| Prop. Glycol | 50% propylene glycol |
| | 50% water |

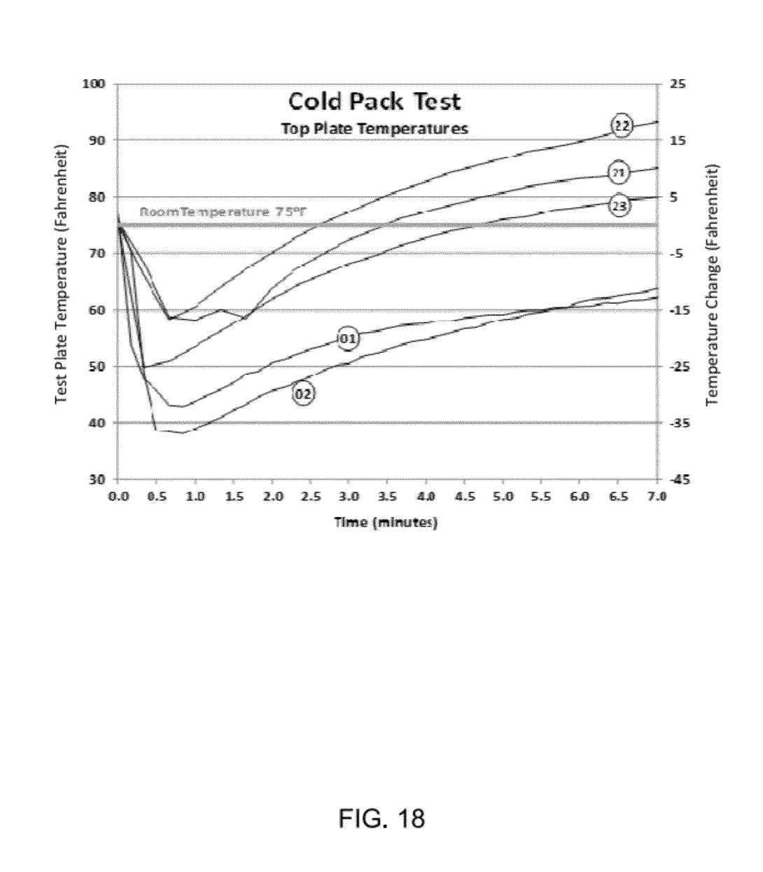

FIG. 18 provides a comparative summary of cold pack performance.

The test data is only provided for illustration of the properties at one medium concentration range with the understanding that modifications to the concentration ranges of the sample components would result in corresponding changes in performance variables which may be calculated by known formulas. The ranges are not provided for limitation of the disclosed device, rather to illustrate exemplary performance properties and enablement. The curves labeled “01” and “02” are exemplary performance curves for samples of the disclosed therapeutic device. The curves labeled “21,” “22,” and “23” are exemplary performance curves for samples of known cold packs and frozen peas.

Example: Hot Pack Cooling Simulation

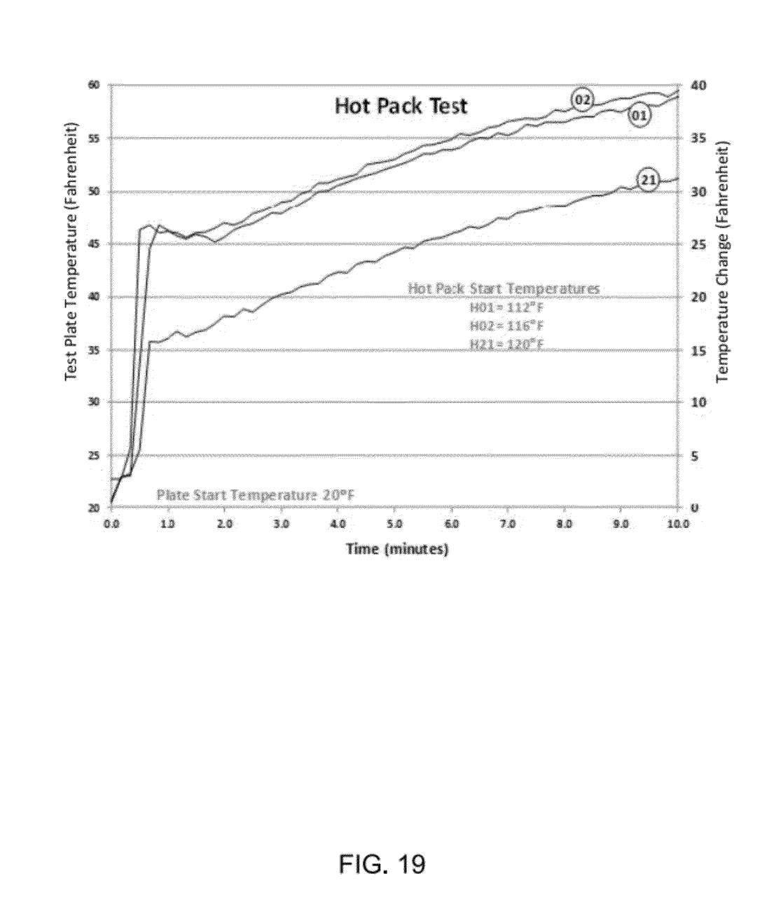

FIG. 19 provides an exemplary comparison of the presently disclosed therapeutic pack as compared to popular therapeutic packs on the market that are also rated for hot and/or cold therapy.

In this comparison, the Samples 1 and 2 are exemplary of the disclosed therapeutic device. The following chart describes the exemplary comparison samples used:

| Sample | Description | Dimensions | Seal Width |
|---|---|---|---|
| 1 | therapeutic device | 5.75″ × 7.4″ | (2x) .46″ (1x) .40″ |
| | according to | | (1x) .3″ |
| | disclosure | | |
| 2 | therapeutic device | 5.8″ × 7.4″ | (2x) .46″ (2x) .37″ |
| | according to | | (1x) .29″ |
| | disclosure | | |
| 21 | Commercial Gel Pack | 4″ × 9.5″ | n/a |
| | 2 (above) | | |

Example

The following data represents other working samples demonstrating the efficacy of the disclosed device to provide heating therapy. This example is merely illustrative and not meant to be limiting.

Experimental Set Up.

The therapeutic packs tested had varying dimensions as shown in the tables below. The therapeutic packs were heated in a 1000W microwave for a given period of time, then placed on foam insulation instrumented with thermocouples.

Peak temperatures for packs heated from room temperature for a given period of time are shown in the following chart:

| Dimensions | MICROWAVE TIME (seconds) | PEAK TEMP. |
|---|---|---|
| 9.0″ × 7.88″ | 60 | 38-43° C. |
| 8.88″ × 7.75″ | 60 | 39-46° C. |
| 8.9″ × 7.75″ | 60 | 41-44° C. |
| 5.75″ × 7.75″ | 60 | 52-57° C. |
| 5.8″ × 7.5″ | 50 | 48-52° C. |
| 2.75″ × 7.25″ | 25 | 44-50° C. |
| 2.75″ × 7.37″ | 25 | 45-49° C. |
| 3.75″ × 5.5″ | 25 | 51-53° C. |
| 3.75″ × 5.5″ | 20 | 44-47° C. |
| 9.0″ × 7.88″ | 70 | 43-45° C. |
| 5.75″ × 7.75″ | 40 | 41-44° C. |
| 5.8″ × 7.5″ | 35 | 39-40° C. |
| 2.75″ × 7.25″ | 20 | 39-42° C. |
| 2.75″ × 7.37″ | 18 | 39-43° C. |
| 3.75″ × 5.5″ | 20 | 43-46° C. |
| 3.75″ × 5.5″ | 18 | 41-44° C. |
| 3.7″ × 5.5″ | 18 | 40-43° C. |
| 3.7″ × 5.5″ | 18 | 39-43° C. |

Although various embodiments of the invention have been described, it will be apparent to those of ordinary skill in the art that many more embodiments and implementations are possible that are within the scope of the invention. For instance, steps of a method as displayed in the figures or reflected in the claims do not require a specific order of execution by way they are presented, unless specified. The disclosed steps are listed as exemplary such that additional or different steps may be executed or the steps may be executed in a different order. Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment.

We claim:

1. A therapeutic device for delivering hot or cold therapy comprising:
an impermeable package;
a plurality of generally spherical solids; and
a fluid medium;
wherein, when cooled to less than −15° C. the medium remains in a fluid phase, and the plurality of generally spherical solids and the medium are contained in the package; and
wherein, the fluid medium and the solids are configured to be heated above body temperature and to be cooled below body temperature for use in therapeutic contact with a body.

2. The therapeutic device of claim 1, the plurality of generally spherical solids comprising glass, high density plastic, hard rubber, or any combination thereof.

3. The therapeutic device of claim 2, the glass generally spherical solids having a diameter of between 4 mm and 14 mm.

4. The therapeutic device of claim 2, the generally spherical solids having a plurality of faceted surfaces or a plurality of indented surfaces.

5. The therapeutic device of claim 2, the generally spherical solids having at least one hole therein.

6. The therapeutic device of claim 1, the medium comprising glycerin.

7. The therapeutic device of claim 1, the medium solution comprising propylene glycol or natural equivalent and water.

8. The therapeutic device of claim 1, the medium comprising at least one of water, alcohol, glycerin, and propylene glycol.

9. The therapeutic device of claim 1, the medium comprising water and propylene glycol in a ratio of at least one of 65:35, 70:30, and 80:20.

10. The therapeutic device of claim 1 being formable to curved contours of the body by virtue of a weight of the generally spherical solids and a viscosity of the medium.

11. An accessory for a therapeutic device comprising:
a therapeutic device according to claim 1;
a pouch for holding the therapeutic device; and
a strap configured to hold the pouch and the therapeutic device on an injured area.

12. The accessory of claim 11, the pouch including a size and shape anatomically specific to provide contact between the therapeutic device and a human user's groin region selected from a male's scrotum for treating post-vasectomy discomfort and a female's perivaginal area for treating post-natal discomfort.

13. The accessory of claim 11, the pouch including a holding area sized to insert the therapeutic device therein and to hold the device in a desired location and orientation.

14. The accessory of claim 11, the pouch further comprising:
a first surface that faces the injured area, the first surface comprising material that assists in regulation of thermal transfer between the therapeutic device and the injured area; and
a second surface that faces away from the first surface.

15. The accessory of claim 14, the first surface comprising mesh fabric, Neoprene, cotton, rubber, or flannel.

16. The accessory of claim 14, the second surface including colors or indicia customized for a user.

17. The accessory of claim 11, wherein the strap comprises an elastic section.

18. The accessory of claim 11, wherein the therapeutic device is configured to form to curved contours of an injured area by virtue of a weight of the generally spherical solids and a viscosity of the medium.

19. The accessory of claim 18, wherein the second surface comprises material that has insulating properties.

20. The accessory of claim 18, wherein the injured area is a scrotum or groin of a user, and the strap includes a portion configured to fit around a waist area of the user.
